(19) United States
(12) Patent Application Publication
(10) Pub. No.: US 2025/0180570 A1
(43) Pub. Date: Jun. 5, 2025

(54) DEVICE AND METHOD FOR ANALYZING KETONES IN BODY FLUIDS

(71) Applicant: ARIZONA BOARD OF REGENTS ON BEHALF OF ARIZONA STATE UNIVERSITY, Scottsdale, AZ (US)

(72) Inventors: Erica FORZANI, Phoenix, AZ (US); Xiaojun XIAN, Sioux Falls, SD (US); Sabrina JIMENA MORA, Chandler, AZ (US); Oscar OSORIO PEREZ, Tempe, AZ (US)

(21) Appl. No.: 18/843,993

(22) PCT Filed: Mar. 3, 2023
(86) PCT No.: PCT/US2023/063726

§ 371 Date: Sep. 4, 2024

Related U.S. Application Data
(60) Provisional application No. 63/316,852, filed on Mar. 4, 2022.

(51) Int. Cl. G01N 33/64; G01N 21/31; G01N 21/78

(52) U.S. Cl. CPC G01N 33/64; G01N 21/31; G01N 21/78

(57) ABSTRACT

A device and method for measuring excreted ketones in a body fluid. The device includes a housing and a sensor positioned within the housing. The sensor includes a body, and a cavity formed in the body, the cavity configured to hold a colorimetric sensing liquid, the body including a ketone permeable medium and a hydrophobic membrane with or without an alkaline material to prevent acidic gases or volatile compound interferents. The sensor is configured to detect presence of ketone in the body fluid in contact with the housing.

### CROSS-REFERENCE TO RELATED APPLICATIONS

This application is a non-provisional of and claims the benefit of U.S. Provisional Application No. 63/316,852, filed on Mar. 4, 2022, the entire contents of which are incorporated herein by reference.

### STATEMENT OF GOVERNMENT INTEREST

This invention was made with government support under R03 EB027336 awarded by the National Institutes of Health. The government has certain rights in the invention.

### TECHNICAL FIELD

The present disclosure is directed to an integrated sensing system to determine ketone production rate and ketone concentrations in body fluids.

### BACKGROUND

The measurement of metabolic rate and acetone levels within the human body is commonly associated with weight management and overall health applications. Metabolic rate can be useful for determining a rate of adenosine triphosphate (ATP) molecules produced at a cellular level to sustain metabolic functions. In some implementations, metabolic rate can be useful for determining a respiratory quotient. The respiratory quotient may be indicative of sources of substrates oxidized at the cellular level to produce ATP (e.g., energy), indicating dominant sources such as carbohydrates, fats, and proteins. Additionally, ketones are biomarkers of fat and protein oxidation. These biomarkers are relevant in weight management and overall health applications. Likewise, ketone bodies are produced in the human liver as a result of lipolysis. Lipolysis causes the release of β-hydroxybutyric acid and acetoacetic acid in the blood and the corresponding release of the decarboxylation byproduct, acetone, in human breath.

Monitoring ketone production in the human body can be useful for several reasons. For example, in patients with type 1 diabetes, an absence of insulin can result in excessive accumulation of ketone bodies in the blood. This state is commonly referred to as ketoacidosis, which lowers blood pH. Ketoacidosis is a potentially life-threatening condition for which patients need to constantly monitor their ketone levels under certain circumstances. Additionally, monitoring ketone levels may be suggested for people who use caloric intake deficit to reduce body weight, or use fat/protein-rich and low carbohydrate diets to achieve a sustained state of higher blood ketone levels. This state is commonly referred to as ketosis. In some cases, diets rich in fat or proteins and low in carbohydrates have been shown to help children with epilepsy to overcome seizures and may be associated with weight loss results and heart congestive failure.

Current approaches used to monitor the state of ketosis include urine dipsticks, electrochemical capillary blood monitors, and breath analyzers. Urine dipsticks are qualitative measurements with poor selectivity towards ketones. Capillary blood measurement may be more reliable for monitoring ketosis and is currently approved method for use both at home and in clinical settings. Although more reliable, blood measurements can be invasive and painful for patients. Breath analysis is considered more acceptable because it is non-invasive and conveniently available, avoiding disturbance to the patient. The dominant ketone in breath is acetone, due to the high volatility of the compound.

Breath acetone is commonly considered a reliable indicator of ketosis and correlates with levels of blood ketones. Some current approaches showing these correlations are performed using gas chromatography (GC) coupled to a chemical identification method and Selected Ion Flow Tube Mass Spectrometer (SIFT-MS). GC lacks real-time analysis capabilities making it a complex tool to use in a clinical setting. SIFT-MS has been used for online real-time analysis of breath samples. Additionally, given the nature of the instrument, SIFT-MS has been successfully used to quantify other gases in breath besides acetone. Some existing commercial breath acetone sensors are too complex or require robust integration with other equipment.

Accordingly, a non-invasive and convenient system to monitor ketone production rate and ketone concentrations in body fluids is desirable.

### SUMMARY

Ketone concentrations are seen as indicators of fat/protein oxidation rates; however, existing approaches do not consider the conditions of sample assessment and simply provide a point-in-time concentration instead of an oxidation rate. Oxidation rate is the parameter of interest because it is more indicative of the body's cellular activity rate to burn adipose tissue or oxidize fat and proteins from diet. To make a comparison, consider an example of physical activity. The current limitations with ketone reporting methods are equivalent to providing a distance walked in an exercise routine, without specifying the time and rate taken to walk. Rate is important because it defines the intensity and level of energy expenditure of the walk exercise. Similarly, ketone production rate indicates the rate of oxidation of fat and proteins which are sources of energy, and therefore, the intensity at which the body is capable to burn of fats and proteins per unit of time (min, hour, day).

The measurement of metabolic rate (kcal/day) and respiratory quotient provide for determination of the rate of oxidation of fat and proteins. However, the respiratory quotient is very sensitive to small changes in diet composition, especially by small amounts of carbohydrates. Therefore, a biomarker that can directly measure the fat and proteins oxidation rate is desirable to complete the overall nutritional and adipose tissue oxidation rates.

In one aspect, the disclosure provides a device for measuring excreted ketones in a body fluid. The device includes a housing and a sensor positioned within the housing. The sensor includes a cavity to hold a colorimetric sensing liquid, the cavity including a ketone permeable medium and a hydrophobic membrane with or without an alkaline material to prevent acidic gases or volatile compound interferents. The sensor detects presence of ketone in the body fluid in contact with the housing.

In some aspects, the hydrophobic membrane and the ketone permeable medium retain the colorimetric sensing liquid separate from the body fluid.

In some aspects, the hydrophobic membrane and the ketone permeable medium hold the sensing liquid stable over time.

In some aspects, the hydrophobic membrane and the ketone permeable medium include a thickness less than about 4,000 micrometers, the thickness provides fast diffusion of a few seconds of ketone therethrough.

In some aspects, the colorimetric sensing liquid includes a volume less than about 1,000 microliters of hydroxylamine acid salt and a pH indicator, iodide-derivative complexes, or amine-derivative diazonium salts.

In some aspects, the housing is transparent.

In some aspects, the device further includes a multiple-wavelength and simultaneous color reader to detect presence of ketone in the body fluid.

In some aspects, the multiple-wavelength and simultaneous color reader includes a CMOS.

In some aspects, the multiple-wavelength and simultaneous color reader measures absorbance quantities from the sensor.

In some aspects, the device further includes a flow sensor or a volume sensor to determine flow rate of the body fluid, volume of the body fluid, and patterns of the body fluid.

In some aspects, the device further includes a temperature sensor to measure temperature of the body fluid.

In some aspects, the device further includes a barometric sensor to measure barometric pressure.

In some aspects, the device further includes a humidity sensor to measure relative humidity of the body fluid.

In some aspects, the device further includes a chemical sensor to sense components present in the body fluid.

In some aspects, the components include oxygen and carbon dioxide.

In some aspects, the device further includes a system to measure metabolic rate and respiratory quotient via oxygen consumption rate and carbon dioxide production rate.

In some aspects, the device further includes a processor to execute a machine learning algorithm to calculate output excreted ketone concentration.

In another aspect, the disclosure provides a method for determining ketone production rate. The method includes exposing a sensor to a body fluid. The method includes sensing, via the sensor, an excreted ketone concentration of the body fluid. The method includes transmitting, via the sensor, a signal indicative of the excreted ketone concentration to an electronic controller. The method includes processing, via the electronic controller, the signal. The method includes determining, via the electronic controller, an excreted ketone production rate based on the processed signal. The method includes providing, via the electronic controller, an output to a user based on the excreted ketone production rate.

In some aspects, determining excreted ketone concentration is based on pre-calibrations performed at single or several wavelengths via a multi-wavelength reader.

In some aspects, the method further includes determining body fluid's excreted ketone production rate at standard conditions.

In some aspects, determining fluid ketone concentration and fluid ketone production rate at standard conditions includes at least one selected from the group consisting of using body fluid patterns, using body fluid excretion rate, and using body fluid volumes.

In some aspects, the method further includes determining, via the electronic controller, a body fluid parameter including at least one selected from the group consisting of an oxygen consumption rate, a carbon dioxide production rate, an acetone production rate, a respiratory quotient, an energy expenditure, and an acetone concentration based on the processed signal. The method further includes providing, via the electronic controller, an output to the user based on the body fluid parameter.

In some aspects, the body fluid is at least one selected from the group consisting of breath, skin, sweat, blood, urine, saliva, or excreted fluid from tissues.

Other aspects of the invention will become apparent by considering the detailed description and accompanying drawings.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

FIG. 1 illustrates a sensor with a liquid sensing probe inside a transparent holding cavity, according to some embodiments.

FIG. 2A illustrates a sensor system including sensor with a reader, according to some embodiments.

FIG. 2B illustrates a device for measuring excreted ketones in a body fluid, according to some embodiments.

FIG. 2C is a graph of visible-range spectral changes of the sensor of FIG. 1 based on exposure of different concentrations of acetone gas, according to some embodiments.

FIG. 2D is a graph of color changes of the sensor of FIG. 1 based on exposure of acetone vapor, according to some embodiments.

FIG. 3A illustrates a sensor with the sensing liquid probe in a compartment, according to some embodiments.

FIG. 3B illustrates a sensor with the sensing liquid probe in a spiral channel, according to some embodiments.

FIG. 3C illustrates a sensor with the sensing liquid probe inside PDMS microspheres (liquid sensing probe-modified spheres) in a compartment, according to some embodiments.

FIG. 3D illustrates a sensor with the sensing liquid probe in a straight stripped channeled structure in a compartment, according to some embodiments.

FIG. 4 illustrates a sensor for ketone skin detection on a flexible PCB with a reader including a thermistor and flexible batteries, according to some embodiments.

FIG. 5 is a graph of an average measured breath acetone concentration from single breathing cycles as a function of breathing cycles during hyperventilation, according to some embodiments.

FIG. 6 is a graph of simulated average breath acetone concentration measured in a single breathing cycle as a function of breathing cycles during hyperventilation, according to some embodiments.

FIG. 7 is a graph of a simulation of breath acetone sample concentration as a function of the volume of a single breath, according to some embodiments.

FIG. 8 is a graph of red, green, and blue spectrum responses from a sensor over time, according to some embodiments.

FIG. 9 is a graph of the colorimetric response of the sensor using a nonlinear Langmuir-like fitting, according to some embodiments.

FIG. 10 is a graph of a sensor response for different sensor parameters, according to some embodiments.

FIG. 11 is a graph of interferent selectivity analysis of the sensor for the acetone detection versus volatile organic compounds (32° C.) at the average human concentration, temperature, and humidity for breath and skin, according to some embodiments.

FIG. 12 is a graph of the calibration of a sensor at two different wavelength ranges, green and blue, according to some embodiments.

FIG. 13 is a graph of the correlation of field tests between the measurements of the sensor and a commercial product to measure blood ketones, according to some embodiments.

FIG. 14 is a graph of the stability test for the sensor over time and under different features and conditions, according to some embodiments.

FIG. 15 is a graph of sensor extracted output concentrations based on signal processing of the sensor absorbance as a function of time, according to some embodiments.

FIG. 16 is a flowchart of a method for determining ketone production rate with a sensor, according to some embodiments.

### DETAILED DESCRIPTION

Before any embodiments of the invention are explained in detail, it is to be understood that the invention is not limited in its application to the details of construction and the arrangement of components set forth in the following description or illustrated in the following drawings. The invention is capable of other embodiments and of being practiced or of being carried out in various ways.

Body fluid excretion rate can be measured with flow or volume sensors. For instance, flow sensors can accurately sense breathing patterns. In some examples, the flow sensors can either provide feedback to the user (e.g., the sample is collected at a constant fluid exhalation flow rate) or analyze a signal provided by the flow sensors to allow for detection of exhaled volume during fluid excretion and fluid excretion rate. In some implementations, detection of exhaled volume during fluid excretion and fluid excretion rate can be combined to provide an absolute amount of ketone produced per unit of time, commonly referred to as ketone production rate (Vket).

Although several current approaches analyze breath acetone, none of the current approaches focus on the concept of ketone production rate. Additionally, none of the current approaches show a total solution towards accurate and robust measurements of acetone based on multiple factors affecting the measurement of breath acetone.

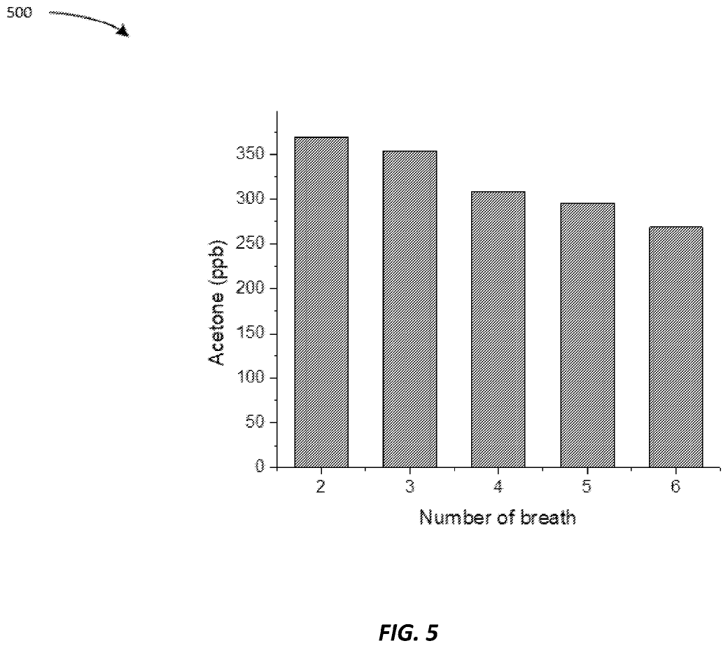

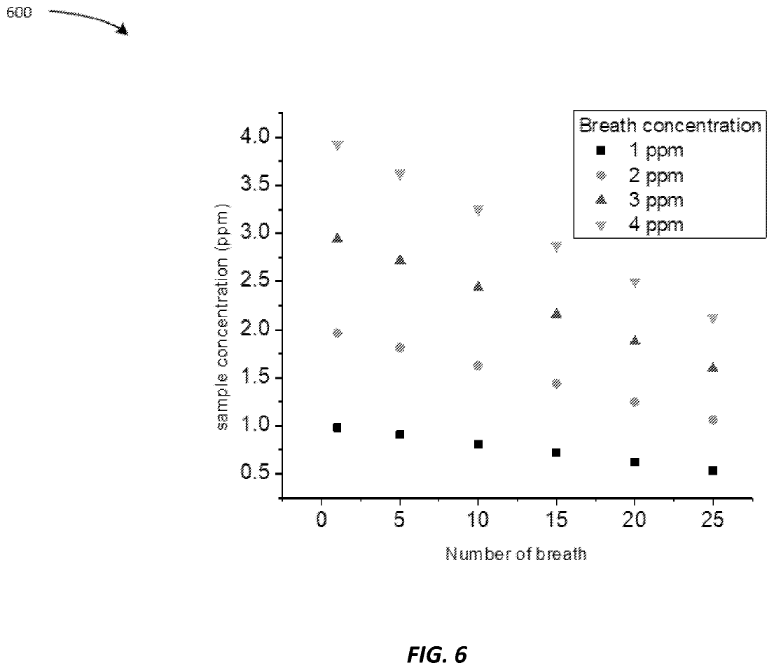

Breath acetone predominantly exchanges in the lung airways. In some instances, human factors such as exhaled air volume, breathing pattern, and breath temperature affect chemical exchange in the lung airways. The human factors should be accounted for when sampling, measuring, and interpreting breath acetone. FIG. 5 is a graph 500 of an average measured breath acetone concentration from single breathing cycles as a function of breathing cycles during hyperventilation, according to some embodiments. As illustrated in FIG. 5, the graph 500 shows an example of how concentration of breath acetone is affected based on previous exhalations. The concentration of acetone decreases with increasing breathing cycles. Therefore, it is expected that breath acetone concentration will increase with breath-holding and decrease with hyperventilation in a manner similar to ethanol. FIG. 6 is a graph 600 of simulated average breath acetone concentration measured in a single breathing cycle as a function of breathing cycles during hyperventilation, according to some embodiments. As shown by graph 600, the simulation considers the contribution of acetone from airways (b) is half of the contribution from alveolar concentration (a), e.g., (b=a/2). Thus, the concentration of acetone decreases with increasing breathing cycles independent of the acetone concentration.

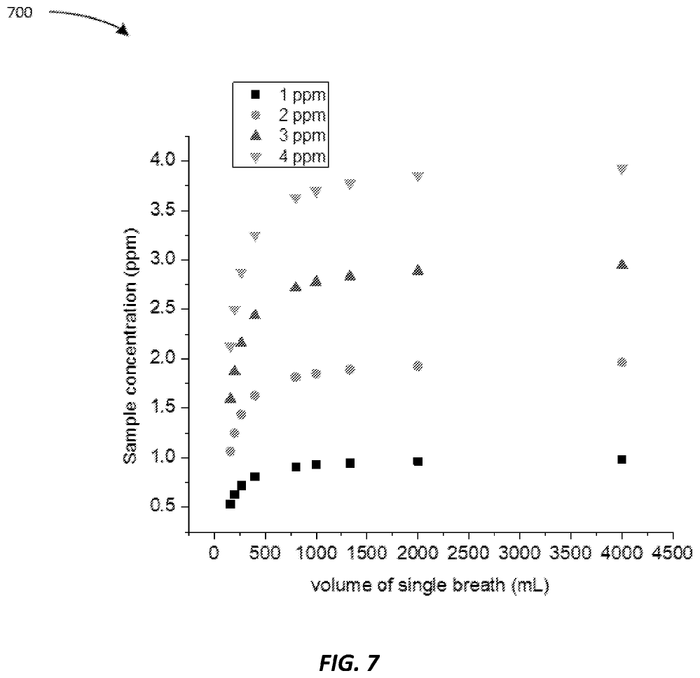

During a single exhalation breath, acetone increases with exhaled volume. Thus, the more air volume exhaled, the greater the acetone concentration. FIG. 7 is a graph 700 of a simulation of breath acetone sample concentration as a function of the volume of a single breath, according to some embodiments. As shown by graph 700, the higher the volume of air exhaled, the more concentration in a single breath. Accordingly, graph 700 illustrates the body fluid's exhalation with respect to the volume of a single breath.

Generally, when providing representative states of fat/protein oxidation independent of assessment conditions, there is a need to normalize body fluid excreted ketone concentrations to excreted flow conditions. Accordingly, reporting of ketone volumes produced per unit of time as ketone production rate is performed. For example, ketone production rate Vket (ml/min) can be defined as follows:

$$Vket ( ml / min ) = Ketone concentration ( volume / sample volume ) \times Sample excretion rate ( sample volume / min ) \qquad Eq . ( 1 )$$

$$or$$

$$Vket ( moles or volume or ppb / {mm}^{2} / min ) = Ketone moles or volume or concentration / [ Area ( {mm}^{2} ) \times collection time ( min )$$

Many current approaches focus on assessing acetone concentrations, but are subjected to the rate of excretion. Additionally, body fluid (e.g., breath) temperature causes an increase in ketone (breath acetone), which indicates the need for detecting the body fluid temperature and other conditions such as pressure and humidity in the fluid to normalize the excreted ketone volumes to standardized conditions such as standard temperature and pressure, dry conditions (STPD). In summary, none of the existing current approaches consider the detection of ketone production rate as an indication of fat/protein oxidation rate, which is very useful when assessing the speed of fat/protein oxidation.

The factors mentioned above impact body fluid ketone differently depending on the manner used to capture the body fluid and portion of excreted body fluid. Therefore, Vket is accounted for the body fluid's ketone excretion rate at standardized conditions, which considers conversion factors from the ketone assessment condition to (for example, but not limited to) STPD as follows:

Conversion from Atmospheric Temperature and Pressure Saturated Condition (ATPS) to Standard Temperature and Pressure Dry Condition (STPD):

$$[ ( {P}_{bar} ( mm Hg ) - {P}_{H 2 O} ( mm Hg ) ) / 760 mm Hg ) ] \times [ \text{} 273 \text{} / ( T ( °C . ) + 273 ) ] \qquad Eq . ( 2 )$$

where P_{bar} is the barometric pressure of the body fluid, P_{H2O} is the partial pressure of body fluid's water, and T is the temperature of the body fluid; all at the point of sample assessment.

In some implementations, excreted body fluid rate can be concurrently tested for temperature (T) and humidity (relative humidity or P_{H2O}), using temperature sensors and relative humidity sensors. The combination of temperature sensors and relative humidity sensors allows the desired absolute excreted ketone concentration and excreted ketone (e.g., acetone) concentration production rate at Standard Temperature and Pressure Dry condition STPD. In some instances, the excreted ketone is acetone, which is detected with a sensor. In some examples, the sensor selectively detects excreted acetone in the presence of other breath gases such as carbon dioxide, water, oxygen, etc.

In some implementations, the sensor (e.g., a ketone sensor) is based on colorimetric detection or fluorescence detection. Both colorimetric detection and fluorescence detection accommodate multi-wavelength readers to satisfy the colorimetric and fluorescence reading conditions. A detection reaction based on a ketone reaction with hydroxylamine acid salt is selective. In some implementations, the detection reaction is applied in the environmental detection of ketones and aldehydes for exposure assessment. The detection reaction is a single-step reaction and avoids the detection of ketones in multiple steps as presented in previous approaches. The single-step detection reaction between acetone and hydroxylamine acid salt results in an acid release causing a local pH change of the environment holding a sensing probe. The pH change is quantified by changing the color of a pH-sensitive dye.

In some implementations, other sensors utilizing a ketone reaction with hydroxylamine acid salt but employing other mechanisms for detecting released acid are provided. In some instances, the sensitivity of a detection setup and the capability of conditioning breath samples for real-time analysis of acetone is disclosed.

In some implementations, a method for acetone detection can be performed by a sensing probe made of a composite of a pH indicator and hydroxylamine acid salt. The sensing probe allows for high accuracy towards the detection of breath, skin, blood, urine, and any body fluid acetone when compared with a gold-standard method of breath, skin, blood, urine and any body fluid acetone, e.g., SIFT-MS methods, which indicates the adequate sensitivity and specificity of the sensing probe.

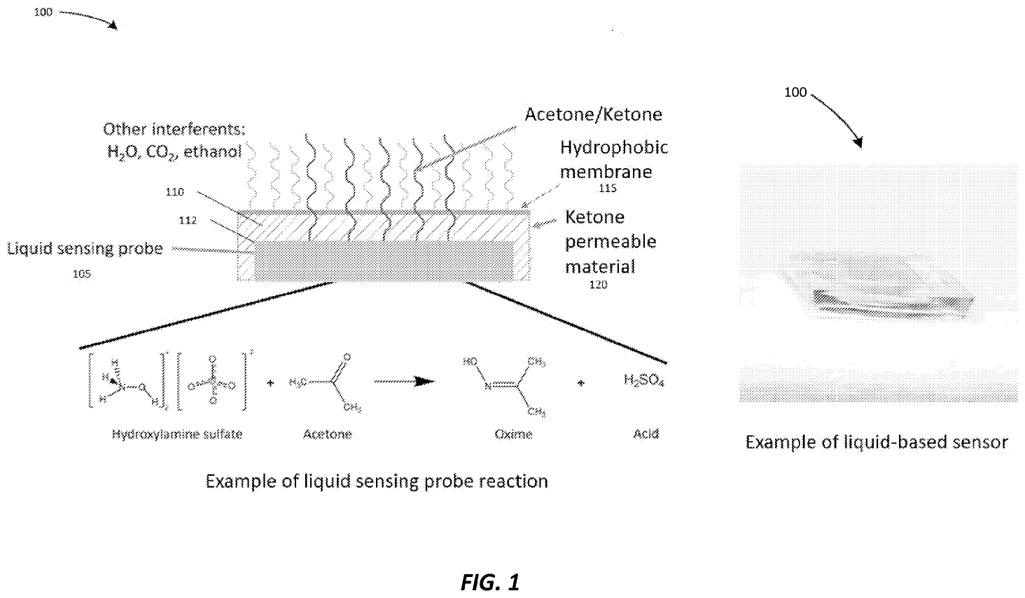

In some examples, a ketone sensor includes a mechanism to hold the sensing probe for ketone in a liquid state (further described below with respect to FIGS. 1-3B). In some implementations, several options of hydroxylamine acid salts are available, such as for example, but not limited to, hydroxylamine sulfate and hydroxylamine hydrochloride. Additionally, several options of pH indicators are available, such as for example, but not limited to, Thymol blue, Bromophenol blue, and Cresol red. Maintaining a sensor configuration in the liquid state allows for the stability to use other commonly used sensing probes for ketones, such as iodine, diazonium salts, etc.

FIG. 1 illustrates a sensor 100 (e.g., a ketone sensor or an acetone sensor), according to some embodiments. The sensor 100 includes a liquid sensing probe 105 and a body 110. The body 110 includes a cavity 112 where the liquid sensing probe 105 is positioned. The body 110 includes a ketone permeable material 120. The body 110 is at least partially surrounded by a hydrophobic membrane 115 (e.g., a hydrophobic layer of polymer). In some embodiments, the body 110 is fully surrounded by the hydrophobic membrane 115. In some embodiments, the hydrophobic membrane 115 is exposed to ketone from a body fluid. In some embodiments, the body 110 may be transparent and may comprise transparent materials, such as transparent polymers. The transparent polymers may include plastic molding materials that are inert to the liquid sensing probe 105. Examples of the transparent polymers include, but are not limited to, polyethylene (PE), polyethylene derivatives (e.g., Polyethylene terephthalate, PET), polypropylene (PP), polydimethylsiloxane (PDMS), and the like.

In some embodiments, the ketone permeable material 120 is exposed to ketone (e.g., acetone) from the body fluid. In such embodiments, the liquid sensing probe 105 detects a presence of ketone in the body fluid when the liquid sensing probe 105 is exposed to the body fluid. In some embodiments, the hydrophobic membrane 115 and the ketone permeable material 120 are configured to retain a colorimetric sensing liquid separate from the body fluid. In some embodiments, the hydrophobic membrane 115 and the ketone permeable material 120 hold the sensing liquid stable over time.

In some instances, the transparent polymers combine synergic properties that contribute to sensor robustness, sensitivity, and specificity. The synergic properties may include, but are not limited to, (i) creating liquid probe holding cavities with volume and dimensions that are appropriate to stably hold a liquid over long periods of time (e.g., avoiding evaporation), (ii) pre-concentrating an analyte (e.g., acetone) from the body fluid onto areas of polymer in contact with the liquid sensing probe 105, (iii) rejecting volatile organic compounds (VOCs) that could act as interferents of the detection reaction, and (iv) providing optimal diffusion thickness to ketones, such as acetone, so that acetone diffuses inside the cavity 112 and reacts with the liquid sensing probe 105. In some examples, the hydrophobic membrane 115 and the ketone permeable material 120 include a thickness less than about 4,000 micrometers. The thickness provides fast diffusion of a few seconds of ketone therethrough. Additionally, transparent polymers offer versatile options to add hydrophobic layers (e.g., the hydrophobic membrane 115) to further protect the liquid sensing probe 105. In some instances, the hydrophobic membrane 115 includes additives such as alkaline chemicals that allow filtering acidic gases or volatile compounds, acting as interferents of the ketone measurement. In some embodiments, the colorimetric sensing liquid within the liquid sensing probe 105 includes a volume less than about 1,000 microliters of hydroxylamine acid salt and a pH indicator, iodide-derivative complexes, or amine-derivative diazonium salts.

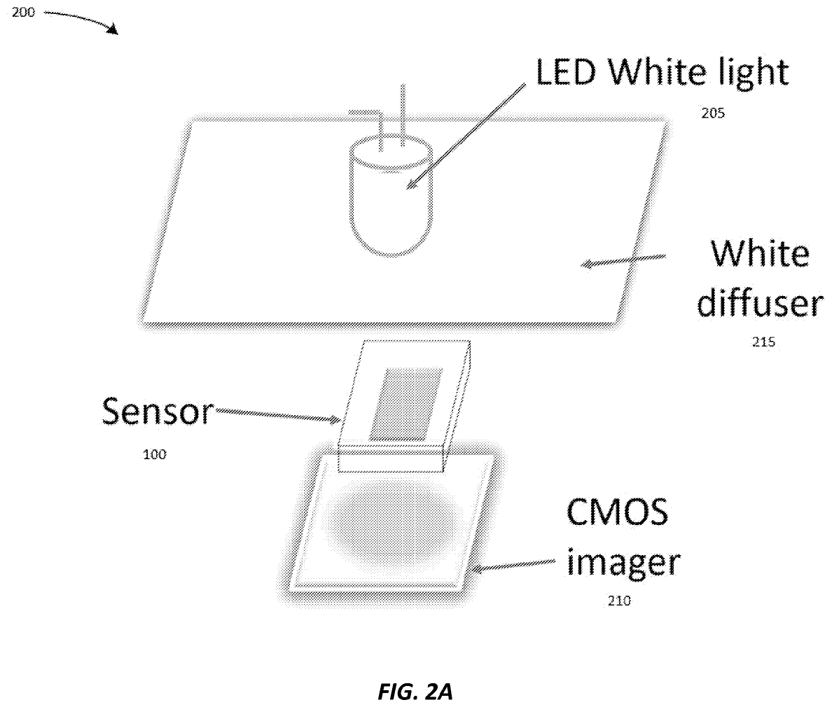

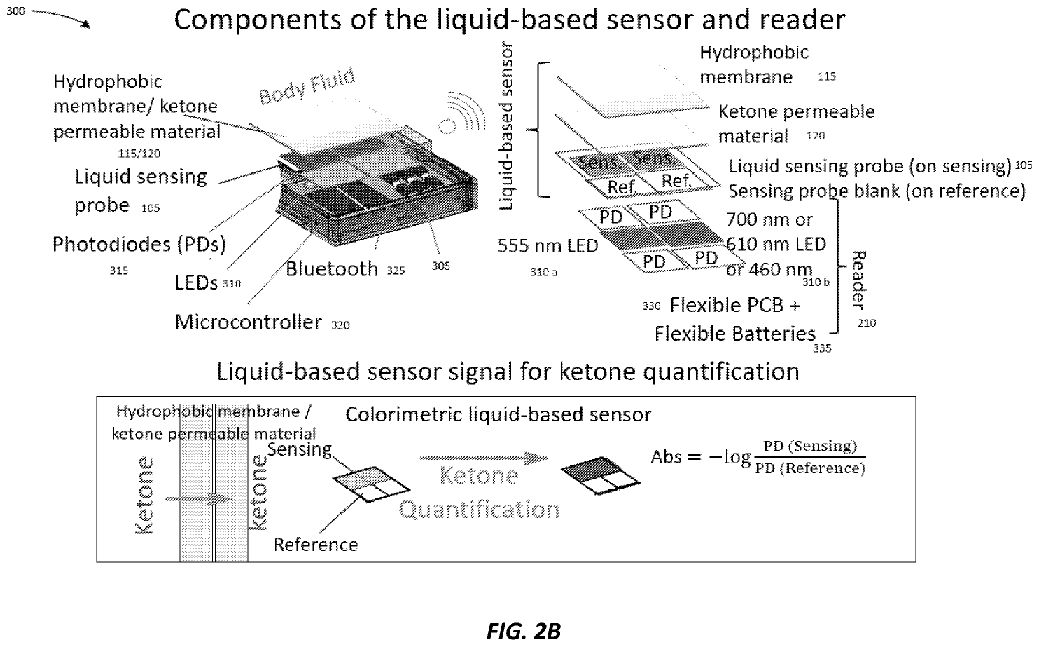

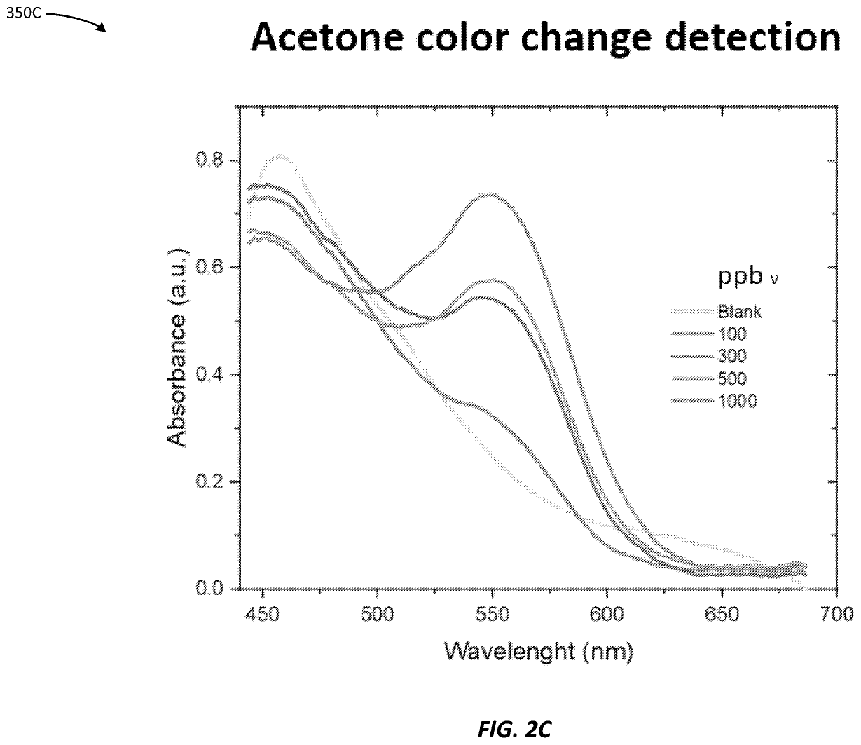

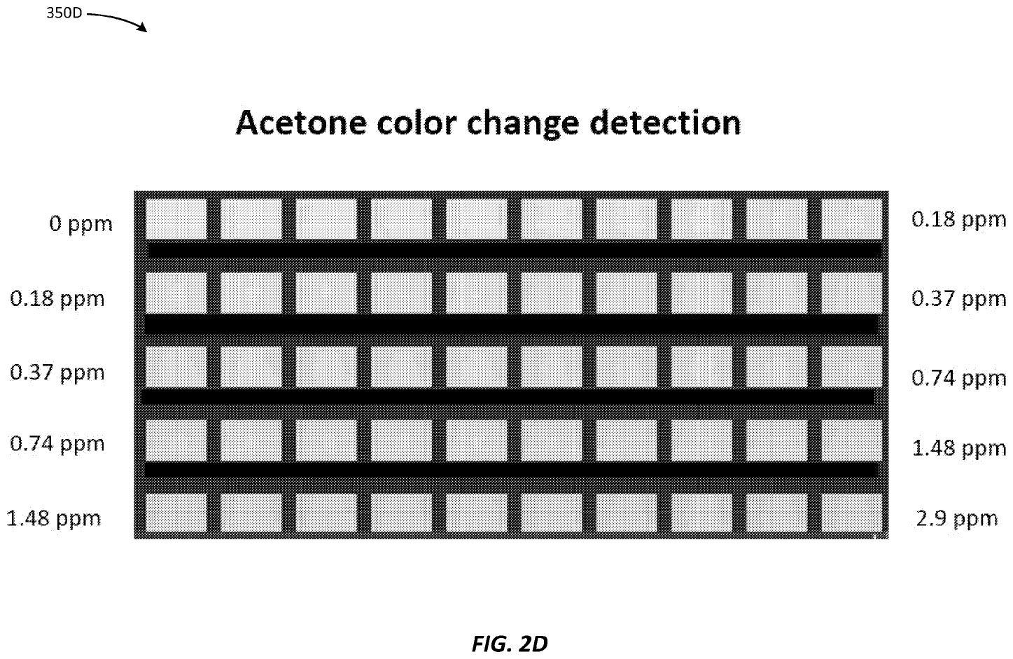

In some embodiments, the sensor 100, including the liquid sensing probe 105 and the cavity 112, may be used with multi-wavelength color/fluorescence sensor readers. FIG. 2A illustrates a sensor system 200 including the sensor 100 with a sensor reader, according to some embodiments. In some embodiments, the sensor 100 is a liquid probe-based acetone sensor. For example, a white light source 205 (e.g., a white light emitting diode (LED)) may be used in combination with a complementary-metal-oxide-semiconductor (CMOS) chip (e.g., a CMOS imager 210). The CMOS imager 210 captures images of the sensor 100 and processes the images to determine a light absorbance or light emission metric for wavelengths in a range of blue, green, and red of the visible spectrum. Based on the light absorbance or light emission, the CMOS imager 210 detects the presence of ketone in the body fluid. In some examples, the sensor system 200 further includes a white light diffuser 215 positioned between the white light source 205 and the sensor 100. The white light diffuser 215 scatters white light provided by the white light source 205 and the scattered white light is captured by the CMOS imager 210. In some examples, the CMOS imager 210 determines a light intensity provided by the white light source 205 through the white light diffuser 215.

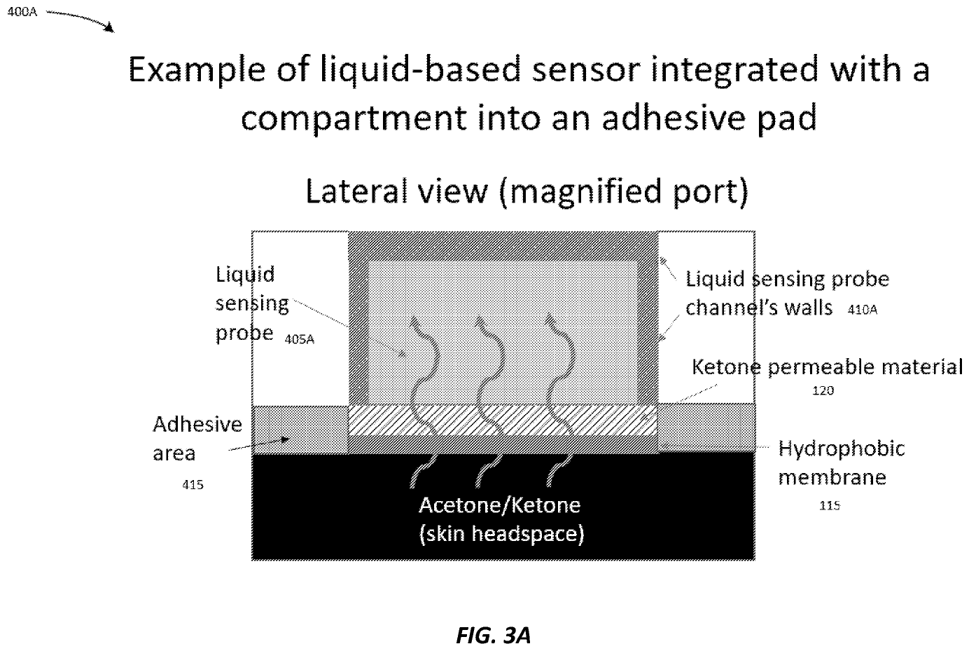

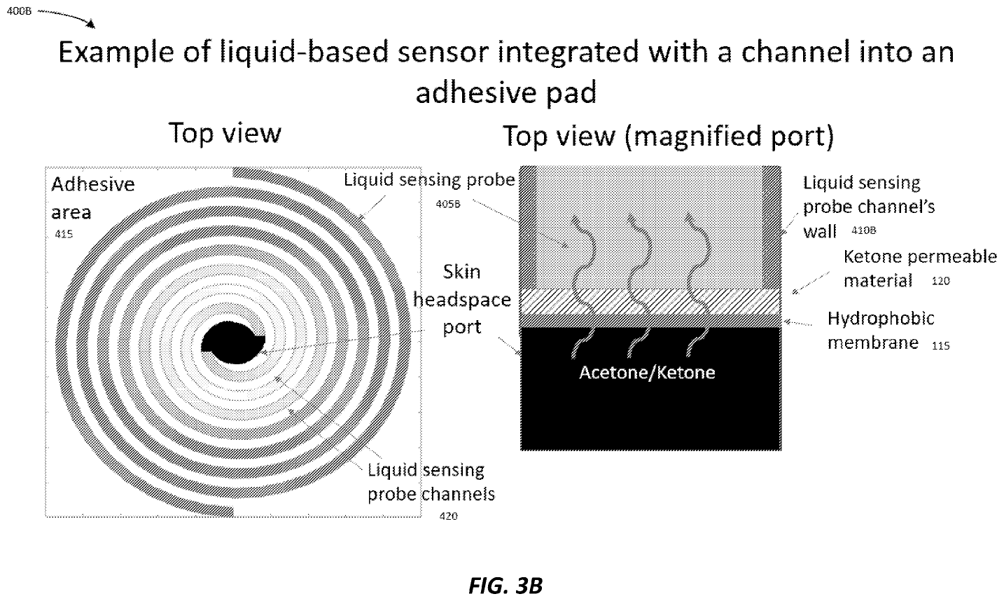

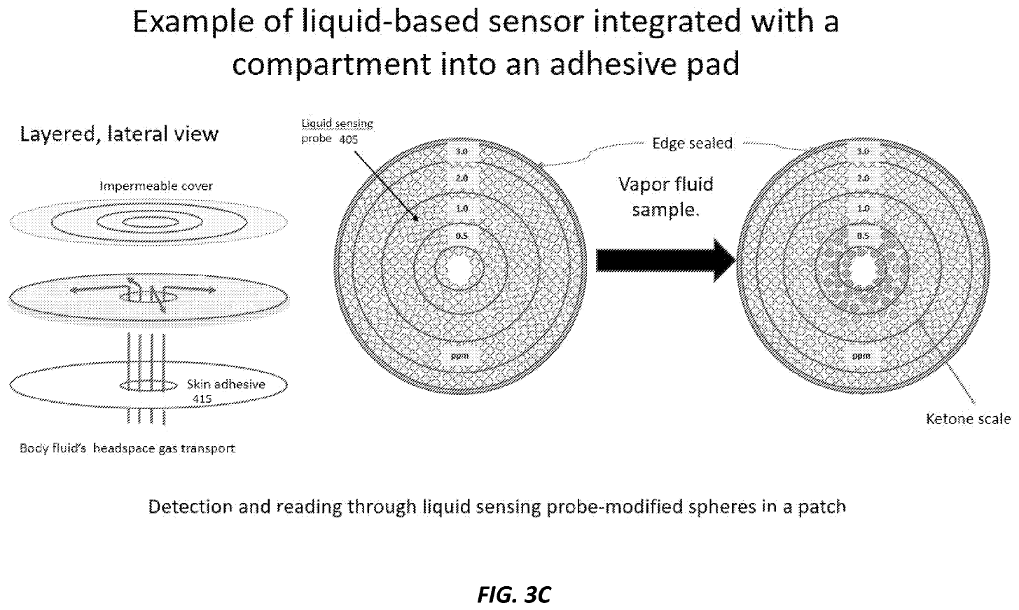

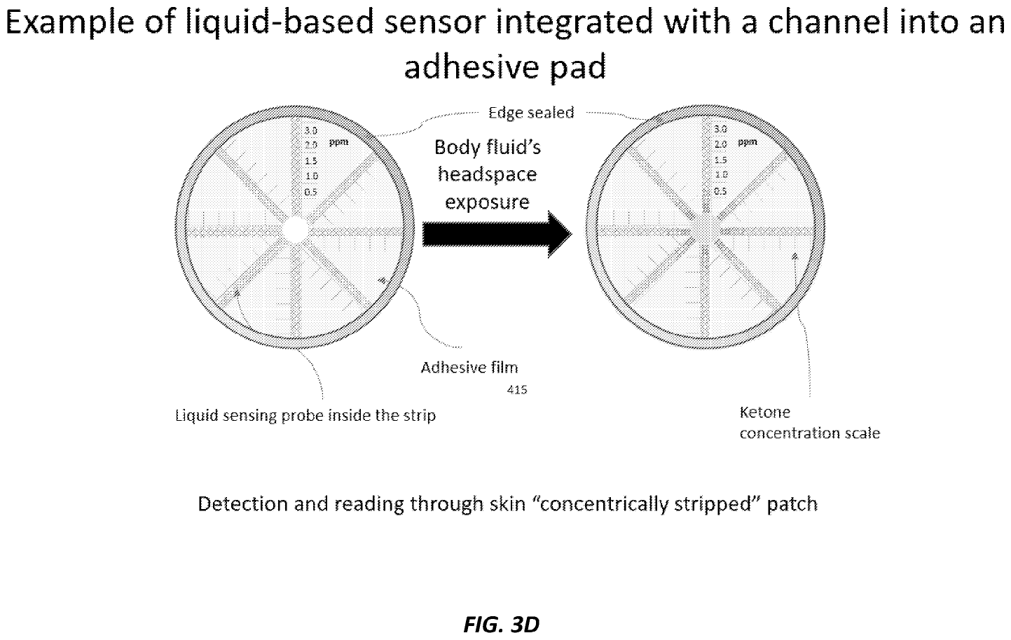

In some embodiments, other multi-wavelength readers can be set to read multiple wavelengths in transmission or reflectance mode by combining different color LEDs and photodiodes (PDs). FIG. 2B illustrates a device 300 for measuring excreted ketones in a body fluid, according to some embodiments. The device 300 includes a housing 305 for holding the various components therein. For example, the sensor 100 may be positioned within the housing 305. As described above, the sensor 100 includes the liquid sensing probe 105, the body 110 with cavity 112, the hydrophobic membrane 115, and the ketone permeable material 120. In some instances, the body 110, the hydrophobic membrane 115, and the ketone permeable material 120 define a sensor housing (further described below with respect to FIGS. 3A and 3B). The cavity 112 includes a colorimetric sensing liquid. In some embodiments, the ketone permeable material 120 and the hydrophobic membrane 115 are manufactured with or without an alkaline material to prevent acidic gases or volatile compound interferents from entering or exiting the cavity 112. The sensor 100 detects a presence of ketone in the body fluid in contact with the sensor housing. In some instances, the device 300 measures metabolic rate and respiratory quotient via oxygen consumption rate and carbon dioxide production rate.

The device 300 further includes a reflectance configuration with multiple LEDs 310 and photodiodes (PDs) 315 to emit light through the sensor 100. In some embodiments, the multiple LEDs 310 include a 555 nanometer (nm) LED 310a and a 700 nm or/and 410 nm LED 310b. For example, FIGS. 2C and 2D provide guidance on wavelength selection rationale. FIG. 2C is a graph 350C illustrating visible-range spectral changes of the sensor 100 with the liquid sensing probe 105 based on exposure of the sensor 100 to different concentrations of acetone gas. For example, as different wavelengths of light are emitted, different amounts of light are absorbed. The sensor 100 may detect this change in color as different concentrations of acetone gas are presented. FIG. 2D is a graph 350D illustrating color changes of the sensor 100 with the liquid sensing probe 105 based on the exposure of acetone vapor from 180 part-per-billion (V/V) concentration to 2.9 part-per-million (V/V) concentration. In some embodiments, the device 300 further includes the CMOS imager 210 and a controller 320. For example, the controller 320 may be a microcontroller, printed circuit board assembly (PCBA), field-programmable gate array (FPGA), or the like. In some embodiments, the controller 320 is programmed to use stored data to control the flow of a breath sample through the sensor 100. In some embodiments, the sensor 100 provides a signal indicative of the presence of ketone in the body fluid to the controller 320. In such embodiments, the controller 320 determines the amount of ketone in the body fluid. In other embodiments, the controller 320 is in electrical communication with the CMOS imager 210 and, in combination with the CMOS imager 210, determines a light intensity provided by the LEDs 310 and photodiodes 315. In some embodiments, the controller 320 includes a processor that executes a machine learning algorithm to calculate output excreted ketone concentration. In some embodiments, the device 300 further includes a Bluetooth module 325. The device 300 may wirelessly communicate with external devices via the Bluetooth module 325. In some embodiments, the CMOS imager 210 may further include a flexible PCB 330 and flexible batteries 335.

Regardless of the multi-wavelength reader configuration, the signal provided by the sensor 100 is read as absolute light intensity (I) or as absorbance, with absorbance at a particular wavelength or wavelength range defined as follows:

$$Δ Signal = - log ( \frac{{I}_{sensing} ( t )}{{I}_{sensing} ( t = 0 )} ) - 1 \qquad Eq . ( 3 )$$

where I_{sensing}(t) is the intensity of the signal at a given time in the presence of the acetone, and I_{sensing}(t=0) is the intensity of the signal at time=0 in the absence of acetone. Alternatively, the sensor signal is also read, using a reference area providing a continuous signal (I_{reference}), which is unexposed to ketone or lacking the sensing probe (for example in FIG. 2B), and be defined as follows:

$$Δ Signal = - log ( \frac{{I}_{sensing} ( t )}{{I}_{reference} ( t )} ) - [ - log ( \frac{{I}_{sensing} ( t = 0 )}{{I}_{reference} ( t = 0 )} ) ] \qquad Eq . ( 4 )$$

In some embodiments, the sensor 100 is integrated as a ketone skin sensor in a device attached to the skin of a patient. In some embodiments, the sensor 100 includes a colorimetric liquid within the liquid sensing probe 105. The colorimetric liquid reacts (e.g., changes color) when exposed to ketones in the body fluid. The sensor 100 senses the reaction of the colorimetric liquid when exposed to ketones within the liquid sensing probe 105. The integrated sensor may include an external or integrated reader. FIG. 3A illustrates a sensor 400A with a liquid sensing probe 405A in a compartment, according to some embodiments. In some embodiments, the sensor 400A includes similar components to the components described above with reference to the sensor 100 of FIG. 1. For example, the sensor 400A may be an embodiment of the sensor 100. Alternatively, FIG. 3B illustrates a sensor 400B with a liquid sensing probe 405B formed as a spiral channel, according to some embodiments. In some embodiments, the sensor 400B includes similar components to the components described above with reference to the sensor 100 of FIG. 1. For example, the sensor 400B may be an embodiment of the sensor 100.

With reference to FIG. 3A, the shape of the sensor 400A containing the liquid sensing probe 405A is complementary to the shape of the compartment. As described above, the liquid sensing probe 405A, the hydrophobic membrane 115, and the ketone permeable material 120 define a sensor housing 410A. In some embodiments, the sensor housing 410A is transparent. In some embodiments, the sensor 400A further includes an adhesive area 415 to secure the sensor 400A to the skin of the patient. A portion of the sensor 400A (e.g., the hydrophobic membrane 115 and the ketone permeable material 120) may be received in a recess of the adhesive area 415.

With reference to FIG. 3B, the sensor 400B containing the liquid-based sensing probe 405B is shaped in the form of a curved continuous channel (e.g., the spiral). As described above, the liquid sensing probe 405B, the hydrophobic membrane 115, and the ketone permeable membrane 120 define a sensor housing 410B. In some embodiments, the sensor housing 410B is transparent. The compartment or the channel include a ketone/liquid sensing probe inert material capable of containing the liquid. With reference to FIG. 3B, the channel can be square-shaped, circular-shaped, trapezoidal-shaped, crown-like, or have any relevant shape for the user. Skin ketones are detected in the liquid sensing probe 405A, 405B either from (i) the bottom of the compartment or the channel provided with the ketone permeable material 120 and the hydrophobic membrane 115 (FIG. 3A), or (ii) one of the ends of the compartment or the channel provided with the ketone permeable material 120 and the hydrophobic membrane 115 (FIG. 3B). Likewise, as described above, the hydrophobic membrane 115 may be further provided with an alkaline material to avoid further interferences from acidic potential gases or volatile organic compounds. In some embodiments, the compartment or the channel include a material capable of containing the liquid and inert to the ketones and liquid sensing probe 405A, 405B. With reference to FIG. 3A, skin ketones are detected in the liquid sensing probe 405A through the diffusion of ketone from the bottom of the compartment (e.g., through the hydrophobic membrane 115 and the ketone permeable membrane 120). With reference to FIG. 3B, skin ketones are detected in the liquid sensing probe 405B through the diffusion of ketone from one of the ends of liquid sensing probe channels 420.

FIGS. 3C and 3D illustrate examples of the sensor 100 for detection of excreted ketones. As shown in FIG. 3C, the liquid sensing probe 405A, 405B is included inside liquid microspheres of polymer permeable to ketones. Accordingly, the liquid sensing probe 405A, 405B detects skin ketones through the liquid microspheres within the adhesive area 415. As shown in FIG. 3D, the liquid sensing probe 405A, 405B is included inside spiral or straight stripped radial shapes. Accordingly, the liquid sensing probe 405A, 405B detects skin ketones through the spiral or straight stripped radial shapes.

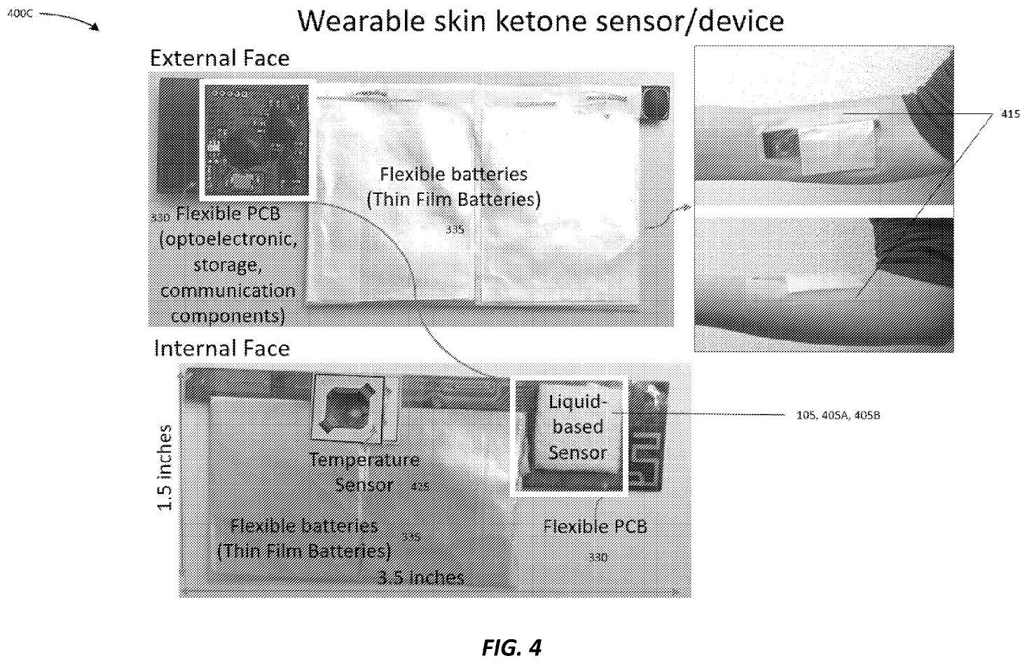

FIG. 4 illustrates a sensor 400C for ketone skin detection on a flexible PCB 330 including the reader with a temperature sensor 425 (e.g., a thermistor) and the flexible batteries 335, according to some embodiments. In some embodiments, the sensor 400C is positioned in direct contact with skin of the patient via the adhesive area 415. In some embodiments, the sensor 400C includes similar components to the components described above with reference to any one of the sensors 100, 400A, or 400B. For example, the sensor 400C may be any one of the sensors 100, 400A, or 400B electrically connected to the flexible PCB 330 and secured to the skin of the patient via the adhesive area 415. In some embodiments, the sensor 400C is integrated to an on-site reader. The sensor 400C is integrated onto the flexible PCB 330 with a multiwavelength reader and multiple sensors, such as the temperature sensor 425. The flexible PCB 330 and the flexible batteries 335 allow for the movement of the sensor 400C and liquid sensing probe 105, 405A, or 405B when in contact with the skin of the patient. In some embodiments, the temperature sensor 425 measures a temperature of the body fluid and transmits a signal indicative of the temperature of the body fluid to the flexible PCB 330. In some embodiments, the flexible PCB 330 includes the controller 320. The flexible PCB 330 determines the temperature of the body fluid based on the signal from the temperature sensor 425. In some embodiments, the sensor 400C includes a flow sensor or a volume sensor to sense flow rate of the body fluid, volume of the body fluid, and patterns of the body fluid. In some embodiments, the flexible PCB 330 determines and provides an output related to oxygen consumption rate, carbon dioxide production rate, acetone production rate, respiratory quotient, energy expenditure, and acetone concentration. In some embodiments, the sensor 400C includes a barometric sensor to measure barometric pressure. In some embodiments, the sensor 400C includes a humidity sensor to measure relative humidity of the body fluid. In some embodiments, the sensor 400C includes a chemical sensor to sense components present in the body fluid. In some instances, the components include oxygen and carbon dioxide.

Existing current approaches do not consider the critical aspect of measuring acetone in connection with body fluid excretion rate, excretion volume, and excretion patterns. In the systems and methods described herein, the detection of body fluid ketone in connection with these factors is focused on. For instance, in the case of breath acetone, the factors include exhalation rate and volumes, breathing patterns, and end-tidal volume. The device 300 determines ketone concentration and ketone production rate via the controller 320 based on the signal provided by the liquid sensing probe 105, 405A, or 405B. Both ketone concentration and ketone production rate are useful for fat and protein oxidation rate evaluation.

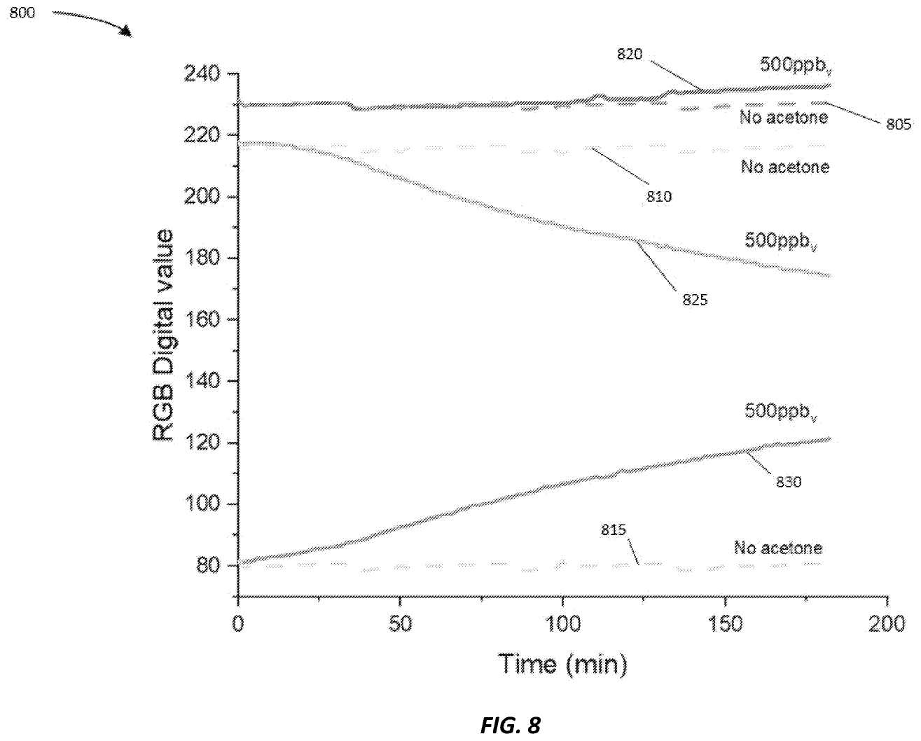

FIG. 8 is a graph 800 of red, green, and blue spectrum responses from a sensor (e.g., a sensor including liquid sensing probe 105, 405A, or 405B) over time using a multi-wavelength reader (e.g., the CMOS imager 210), according to some embodiments. Dashed lines represent the response of the sensor in the absence of acetone. For example, dashed line 805 is the red spectrum response in the absence of acetone, dashed line 810 is the green spectrum response in the absence of acetone, and dashed line 815 is the blue spectrum response in the absence of acetone. Solid lines are the sensor response to [500 ppbᵥ] acetone. For example, line 820 is the red spectrum response to 500 ppbᵥ acetone, line 825 is the green spectrum response to 500 ppbᵥ acetone, and line 830 is the blue spectrum response to 500 ppbᵥ acetone. As shown by graph 800, the green spectrum and the blue spectrum rendered the highest changes of absorbance signal with the presence of acetone.

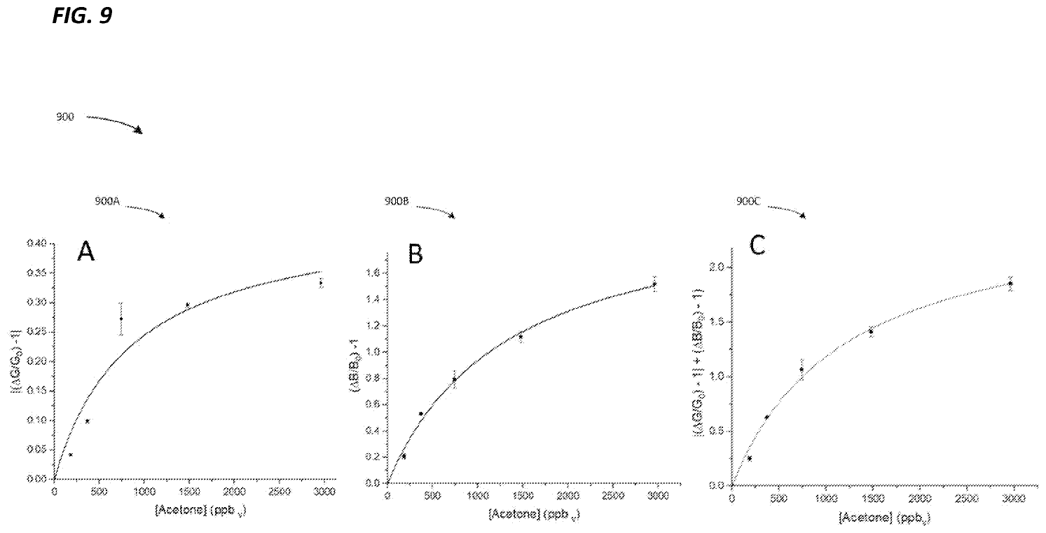

A sensitivity of the sensor (e.g., a sensor including liquid sensing probe 105, 405A, or 405B) may be evaluated and pre-calibrated by exposing the sensor to different concentrations of the acetone in the absence and presence of potential interferences, and simulated or real samples. FIG. 9 is a graph 900 of the colorimetric response of the sensor (e.g., a sensor including liquid sensing probe 105, 405A, or 405B) using a nonlinear Langmuir-like fitting, according to some embodiments. The sensors were exposed to different acetone concentrations (100-3000 ppbᵥ) at a temperature of 32° C. (e.g., breath temperature). The responses are shown as normalized response for intensities captured at (i) Green (G) shown by graph 900A, (ii) Blue (B) shown by graph 900B, and (iii) Absolute Green and Blue wavelengths shown by graph 900C. Gₒ and Bₒ represent the intensities of the sensor recorded at time=0 for the green and blue wavelengths. The Langmuir equation was used to fit the sensor response, with a resulting squared-correlation coefficient (R²) of >0.99. It should be understood that each concentration represents the averaged signal from three independent sensors (e.g., multiple liquid sensing probes). The standard deviation of the average is shown as error bars. As shown in FIG. 9, the sensor is exposed to low concentrations of acetone, which are typically found in breath or skin or headspace of liquid body fluids such as blood, saliva, and urine. As shown in FIG. 9, the sensor has a response with significant sensitivity in the excreted acetone detection range. Additionally, the response obtained from independent sensors has low dispersion (shown in error bars as standard deviations), low dispersion is indicative that the sensors can be fabricated reproducibly.

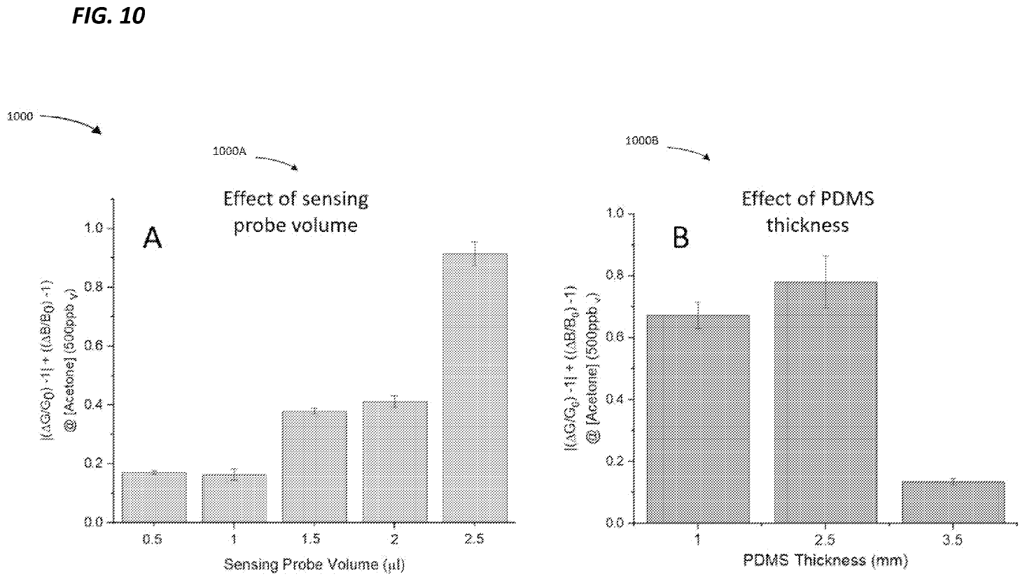

In some implementations, the sensitivity of the sensor is tuned to different acetone concentration ranges by optimizing the sensor's parameters. Some parameters include the volume of the liquid sensing probe 105, 405A, or 405B inside the sensor and the thickness of the diffusional barrier between the body fluid (gas phase) and the liquid sensing probe 105, 405A, or 405B. FIG. 10 is a graph 1000 of a sensor response for different sensor parameters, according to some embodiments. For example, graph 1000A shows the effect of different volumes of liquid sensing probe 105, 405A, or 405B on the sensor sensitivity. As shown by graph 1000A, a volume of liquid sensing probe of 2.5 μL increases the sensitivity by 2-folds with respect to 1.5-2.0 μL and by over 4-fold with respect to 0.5-1 μL. In another example, graph 1000B shows that increasing the thickness of the membrane separating the liquid sensing probe 105, 405A, or 405B from the body fluid, may increase the sensitivity if the membrane has pre-concentrating properties for acetone (as it is known for PDMS). In some instances, the increase of thickness may also increase the diffusional barrier for acetone diffusion into the liquid sensing probe 105, 405A, or 405B. Therefore, an optimal thickness defines the maximum sensitivity of the sensor.

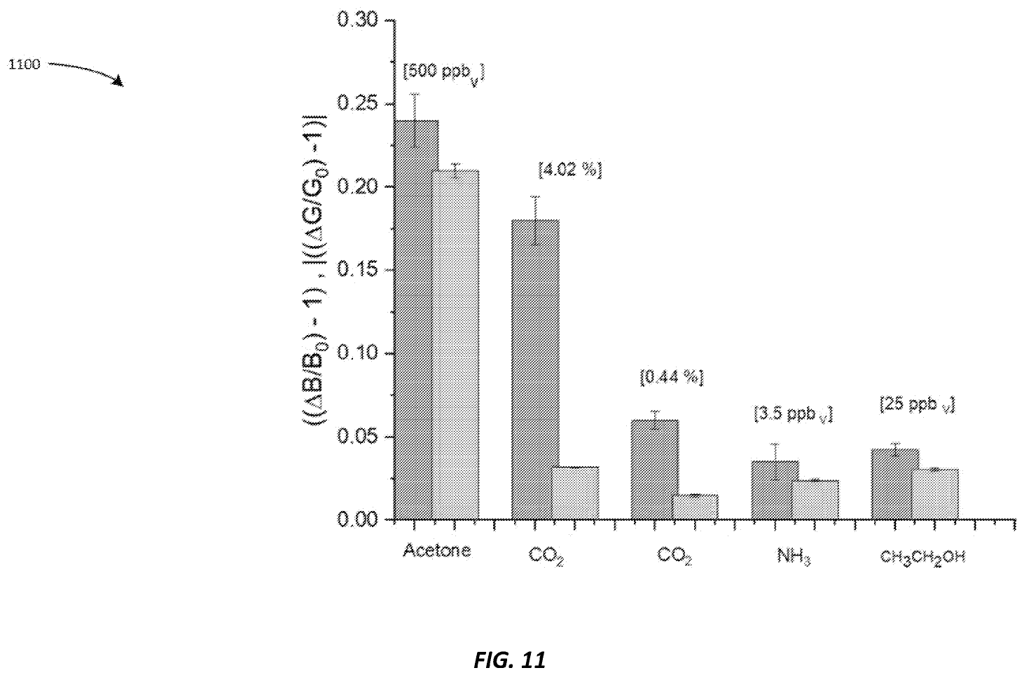

In some embodiments, the sensor (e.g., a sensor including liquid sensing probe 105, 405A, or 405B) is selective to the acetone response and selectively calibrated for detecting acetone in a particular type of body fluid, including breath, skin, blood, saliva, urine. FIG. 11 is a graph 1100 of interferent selectivity analysis of a sensor for the acetone detection versus volatile organic compounds (32° C.) at the average human concentration, temperature, and humidity for breath and skin, according to some embodiments. The blue intensity contribution (e.g., the left bar in each pair of bars) and the green intensity contribution (e.g., the right bar in each pair of bars) to the total signal selectivity is indicated. For CO₂, 4% and 0.4% represent typical concentrations of the gas in breath and skin, respectively. For ammonia, the contribution from the skin ammonia is represented. For ethanol, the contribution from the alcohol-free breath is represented. From all interferents tested, carbon dioxide presented a crossed response with the sensor that could be incorporated into a pre-calibration method established via the multi-wavelength reader.

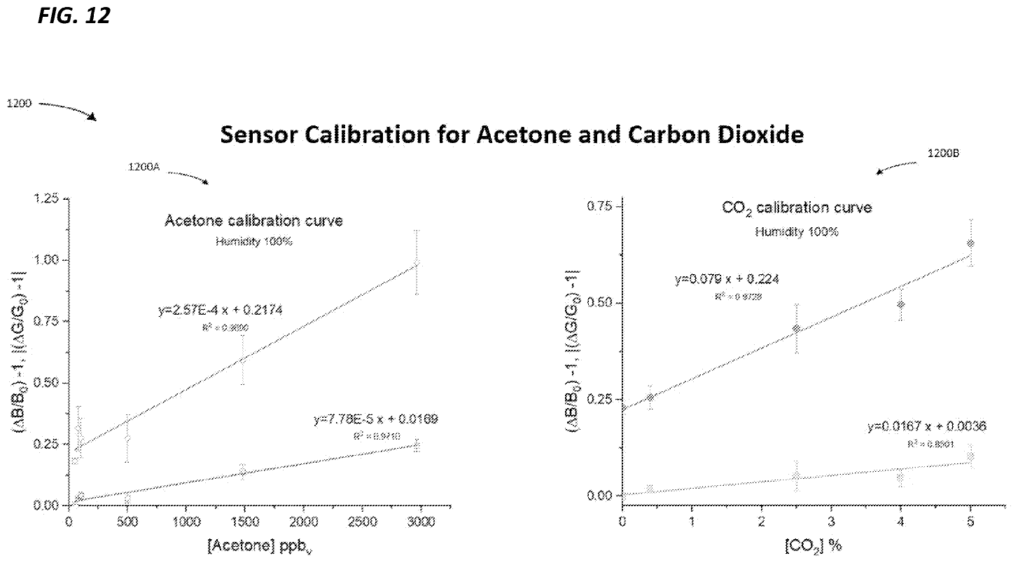

FIG. 12 is a graph 1200 of the calibration of a sensor (e.g., a sensor including liquid sensing probe 105, 405A, or 405B) at two different wavelength ranges, green and blue, according to some embodiments. The calibration for different acetone levels (e.g., graph 1200A) and carbon dioxide levels (e.g., graph 1200B) is performed in a humid environment to simulate fluid body conditions of breath and skin. The pre-calibration allows reading of acetone concentrations with the sensor. In some embodiments, systems and methods described herein determine excreted ketone concentration with the pre-calibrations performed at single or several wavelengths. Additionally, the pre-calibration factor can be incorporated in QR code images, which is useful for importing calibration factors into algorithms with corresponding equations to read acetone concentrations in real samples of body fluids.

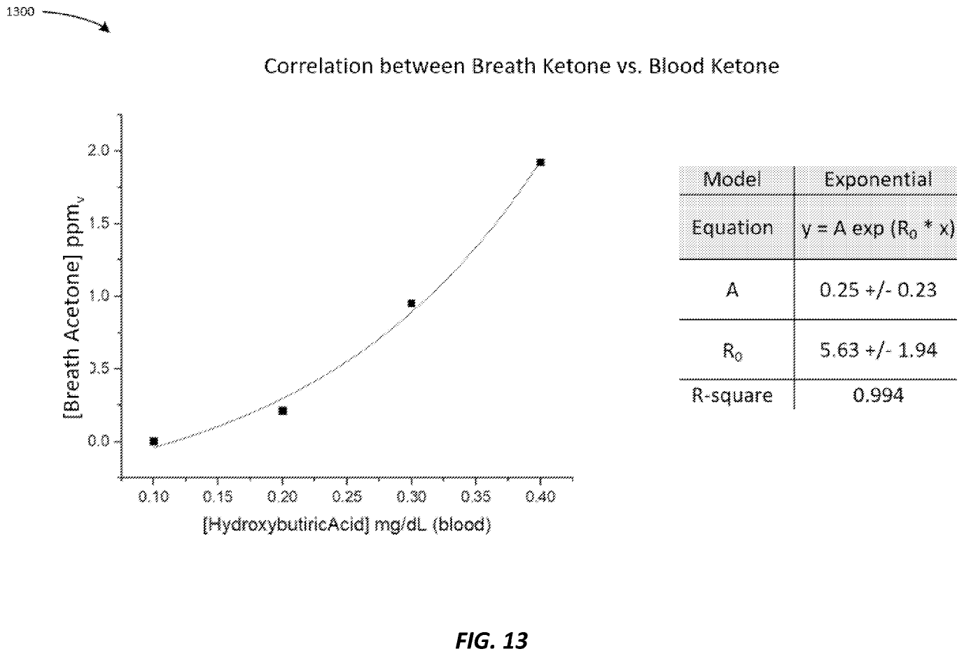

In some embodiments, the pre-calibration algorithms are used to extract acetone concentrations. FIG. 13 is a graph 1300 of the correlation of field tests between the measurements of the sensor (e.g., a sensor including liquid sensing probe 105, 405A, or 405B) and a commercial product to measure blood ketones and hydroxybutyric acid of subjects, according to some embodiments. Blood ketones were measured using Precision Xtra™ electrochemical capillary blood monitor from Abbott. Standard procedure described with the monitor was used to test the samples. As shown in graph 1300, the breath acetone levels correlate with the ketone level detected in blood.

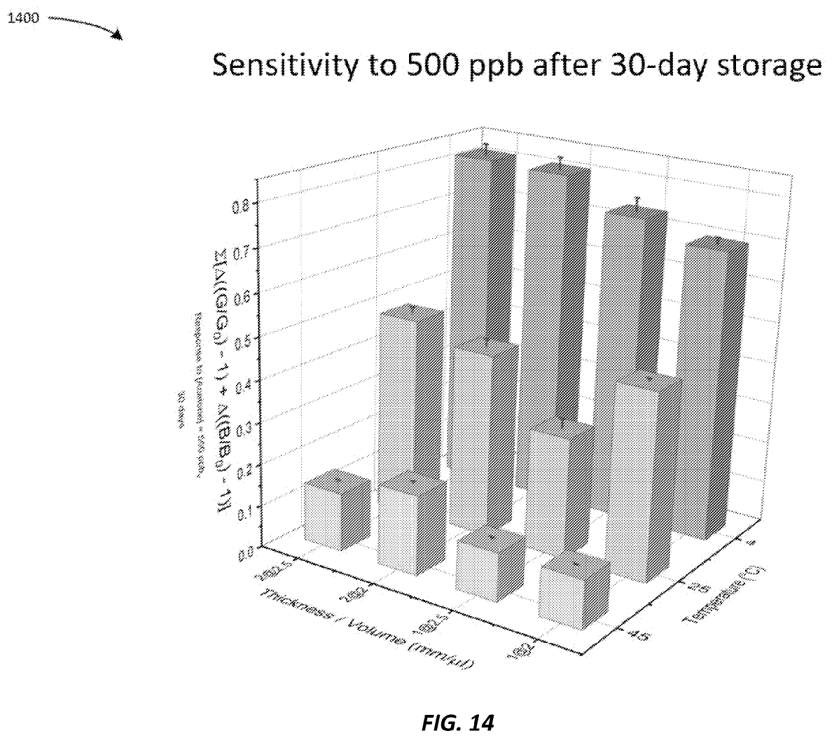

The sensor (e.g., a sensor including liquid sensing probe 105, 405A, or 405B) shows stability toward the sensitive detection of acetone for long periods of time when stored at appropriate temperatures. FIG. 14 is a graph 1400 of a stability test for the sensor over time and under different features and conditions, according to some embodiments. Graph 1400 shows the stability of four different sensor arrays stored at three different temperatures: 4, 25, and 45° C. In some examples, variations are made in the thickness and volume of the liquid sensing probe. For example, the arrangements were 1 mm and 2 mm thick with 2 and 2.5 microliters, respectively. The thickness of the PDMS is controlled by the weight in grams in a mold. After one month, the response of the sensors was evaluated and compared with the original values (day 1 of manufacturing). As shown in FIG. 14, it can be found that the sensor maintains stability for a month. Further, the sensors have been tested and proven to be robust when stored for over a year (not shown). As shown by graph 1400, the sensor that showed the best response for acetone detection by color intensity was the sensor with 2 mm PDMS thickness, 2.5 microliters of liquid sensing probe solution, and stored at 4° C. for a period of one month. Graph 1400 shows the response when exposing the sensor to acetone at a concentration of 500 ppb.

The stability mentioned above is achieved by the capacity to maintain the sensing probe for ketone in a liquid state for an extended period of time, which includes the ability to choose an appropriate housing and also the ability to pack the sensor under pristine conditions with control air quality for humidity, oxygen, carbon dioxide, volatile organic compounds and other gases typically present in the environment. The stability mentioned above can also be achieved because a sensor pre-conditioning process is applied with accelerated thermal aging before the sensor calibration. The results mentioned above indicate that sensors (e.g., a sensor including liquid sensing probe 105, 405A, or 405B) are robust over time, sensitive to low concentrations of acetone, and when combined with the correct multi-wavelength reader, a pre-calibration procedure can allow the sensor to detect acetone in body fluids accurately.

As described above, acetone is a fat-oxidation metabolite. The systems and methods described herein allow for the passive detection of acetone excreted in the skin. The sensor 100 (or similar sensor configurations described herein) is placed on the body and readings are retrieved on demand every 24 hours to obtain daily excreted acetone average (ppm/cm2/24 hours), as well as daily fat burning (g fat/day). In some embodiments, the sensor 100 is part of a wearable device (e.g., sensor 400C) with an adequate optoelectronics system including LEDs 310 in the green, blue and/or infrared wavelength and photodetectors 315, either in a transmission or reflectance configuration. The optoelectronic system could also have an LED 310 and a CMOS imager 210 for deconvolution of light intensity components (Red, Green, and Blue), either in a transmission or reflectance configuration. The sensor and the wearable device are in contact with the skin to passively and non-invasively measure the acetone emitted by the skin.

In some embodiments, the sensor 100 may be inserted into a wearable device with the optoelectronics system so that it can be worn, for example, on the person's arm. One portion of the sensor 100 is placed in contact with the skin and the device's optoelectronics system reads the concentration of acetone. In some embodiments, the optoelectronics system (e.g., including the controller 320) sends the information to a smartphone application to report the acetone concentration to the user.

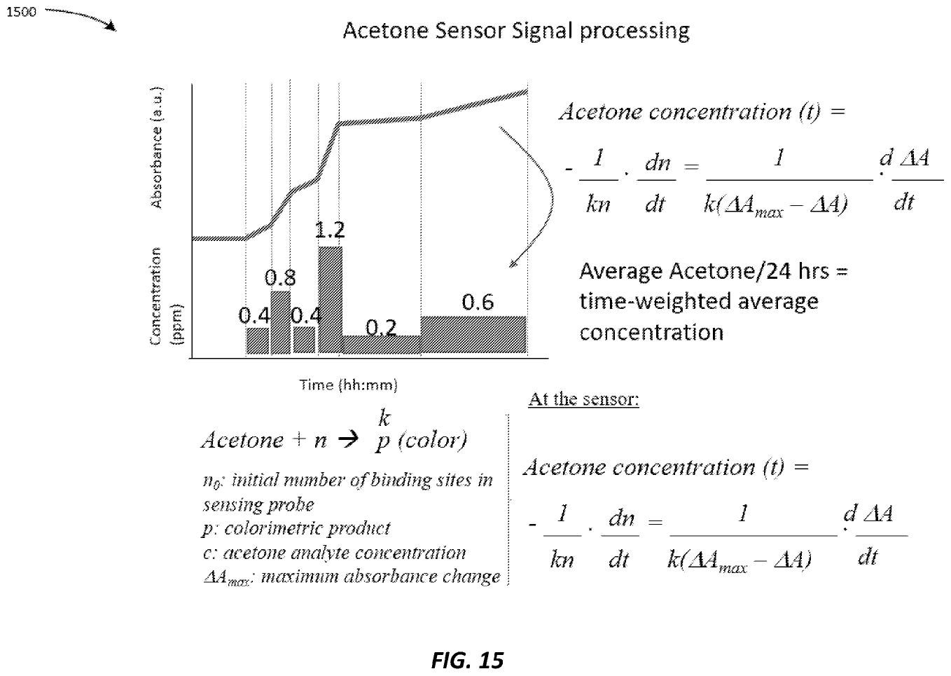

In some embodiments, the color change of the sensor is measured as absorbance signal or similar signals. For example, the sensor signal may be processed as: −log (Signal from the sensing probe area)/(signal from the reference area without sensing probe). The sensor materials (e.g., liquid sensing probe 105, 405A, or 405B) can be adjusted to provide an absorbance signal (or similar signal) that changes linearly over time upon exposure to an acetone concentration. In some instances, the absorbance change (or similar signal) over time (e.g., delta Absorbance/delta Time) is directly proportional to the acetone and the sensor signal (absorbance vs. time) can provide an indication of the acetone concentration by using equations like the equation shown in the FIG. 15. FIG. 15 is a graph 1500 of sensor extracted output concentrations, according to some embodiments. For example, the sensor extracted output concentrations may be determined from a delta Absorbance/delta time signal and time-weighted averaged concentrations. Based, on sensor 100, the concentration changes within the sensor 100 depend on the rate of diffusion of the analyte through the encapsulating material of the sensor, the rate of excretion of acetone through the skin, and the reaction of the sensing solution with acetone.

For example, the sensor signal is analyzed so that the sensor 100 transmits output concentration of acetone for periods of time when the delta absorbance/delta time is constant (e.g. 3 hours or more). Accordingly, the concentrations are time-weighted and averaged to report acetone average in 24 hours as shown in FIG. 15.

Since the sensor signal processing relay on conditions of unsaturated sensor signal, an absorbance change threshold is determined to alert the sensor user to change the sensor 100. Accordingly, the sensor 100 provides a response under unsaturated conditions. For instance, for a medium ketosis stage with acetone average of 1 ppm in 24 hours and a total absorbance change of 0.25 absorbance units in 24 hours, the sensor 100 would last 8 days, if (i) the sensor 100 works under unsaturated conditions and a linear response in a range of absorbance from 0.0 to 2.0, and (ii) the patient maintains a medium ketosis stage ˜1 ppm during 8 days. Alternatively, if the patient has a high level of ketosis of 3 ppm in 24 hours, then, the same sensor configuration would last 2.7 days.

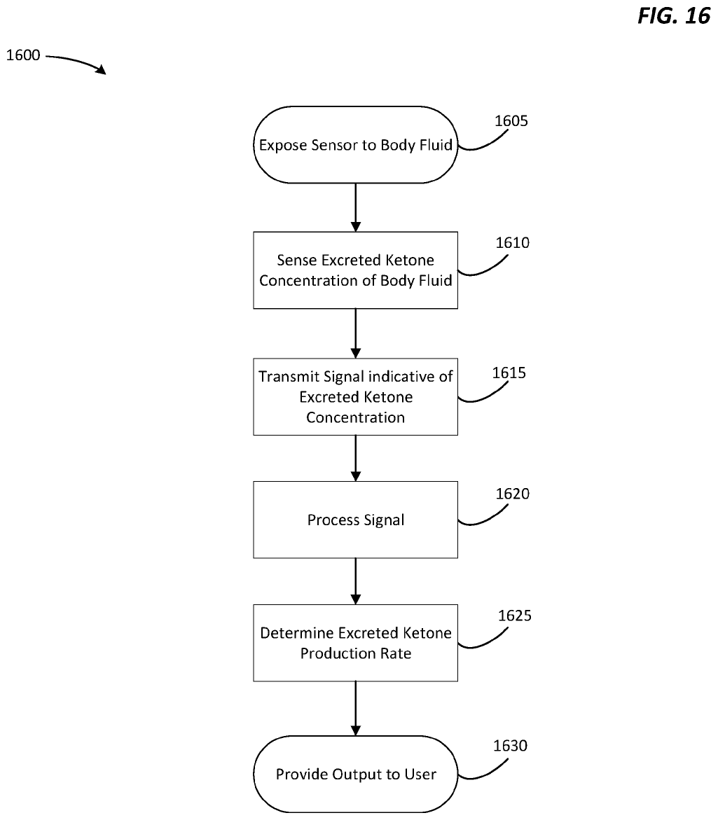

FIG. 16 is a flowchart illustrating a method 1600 for determining ketone production rate using the sensor 100, according to some embodiments. Although the method 1600 is described as using the sensor 100, it should be understood that the sensor 400A, 400B, or 400C may be used to implement the method 1600. It should be understood that the order of the steps disclosed in the method 1600 could vary. For example, additional steps may be added to the process and not all of the steps may be required, or steps shown in one order may occur in a second order. The method 1600 begins at step 1605 when the sensor 100 is exposed to the body fluid. For example, the sensor 100 is attached to the patient or patient's body fluid sample, and is exposed to ketones from the body fluid. For example, the body fluid is breath, skin, sweat, blood, urine, saliva, or excreted fluid from tissues. The method 1600 then proceeds to step 1610.

At step 1610, the sensor 100 senses an excreted ketone concentration of the body fluid. For example, when exposed to ketones from the body fluid, the liquid sensing probe 105 (including the colorimetric liquid) reacts to the ketones based on the excreted ketone concentration. The method 1600 then proceeds to step 1615. At step 1615, the sensor 100 transmits a signal to the controller 320 indicative of the excreted ketone concentration. Alternatively, the CMOS imager 210 or LED—photodiode assembly may transmit the signal indicative of the excreted ketone concentration to the controller 320. The method 1600 then proceeds to step 1620.

At step 1620, the controller 320 processes the signal indicative of the excreted ketone concentration. For example, the controller 320 determines the excreted ketone concentration by implementing equations 3, or 4, alone or in combination with the equation shown in FIG. 15, as described above. In some examples, the controller 320 executes a machine learning algorithm to calculate output excreted ketone concentration. In some examples, the controller 320 determines the excreted ketone concentration based on pre-calibrations performed at single or several wavelengths as described above with reference to FIGS. 11-13. The method 1600 then proceeds to step 1625. At step 1625, the controller 320 determines an excreted ketone production rate based on the excreted ketone concentration and other measured parameters (e.g., time, excretion area, sample excretion rate or flow, and temperature, pressure, and humidity conditions). For example, the controller 320 determines the excreted ketone production rate by implementing equation 1 as described above. In some embodiments, the controller 320 determines the excreted ketone production rate based on Standard Temperature and Pressure Dry conditions (STPD). For example, the controller 320 uses body fluid patterns, body fluid excretion rate, or body fluid volumes at standard conditions to determine the excreted ketone production rate and the excreted ketone concentration. In some embodiments, the controller 320 also determines a body fluid parameter. The body fluid parameter may include an oxygen consumption rate, a carbon dioxide production rate, an acetone production rate, a respiratory quotient, an energy expenditure, and an acetone concentration based on the processed signal. The method 1600 then proceeds to step 1630.

At step 1630, the controller 320 provides an output to the user. For example, the controller 320 provides an output indicative of the excreted ketone production rate to the user via a display (not shown) of the device 300. In other examples, the controller 320 provides an output indicative of the excreted ketone production rate to an external device via the Bluetooth module 325. In some embodiments, the controller 320 also provides an output to the user based on the body fluid parameter. It should be understood that the method 1600 may be performed multiple times to determine consecutive excreted ketone production rates.

Thus, the disclosure provides, among other things, a device and method for analyzing ketones in body fluids. Various features and advantages of the invention are set forth in the following claims.

What is claimed is:

1. A device for measuring excreted ketones in a body fluid, the device comprising:
a housing; and
a sensor positioned within the housing, the sensor including
a body,
a cavity formed in the body, the cavity configured to hold a colorimetric sensing liquid, the body including a ketone permeable medium and a hydrophobic membrane with or without an alkaline material to prevent acidic gases or volatile compound interferents,
wherein the sensor is configured to detect presence of ketone in the body fluid in contact with the housing.

2. The device of claim 1, wherein the hydrophobic membrane and the ketone permeable medium are configured to retain the colorimetric sensing liquid separate from the body fluid.

3. The device of claim 1, wherein the hydrophobic membrane and the ketone permeable medium are configured to hold the sensing liquid stable over time.

4. The device of claim 1, wherein the hydrophobic membrane and the ketone permeable medium include a thickness less than about 4,000 micrometers.

5. The device of claim 1, wherein the colorimetric sensing liquid includes a volume less than about 1,000 microliters of hydroxylamine acid salt and a pH indicator, iodide-derivative complexes, or amine-derivative diazonium salts.

6. The device of claim 1, wherein the housing is transparent.

7. The device of claim 1, further comprising a multiple-wavelength and simultaneous color reader to detect presence of ketone in the body fluid.

8. The device of claim 7, wherein the multiple-wavelength and simultaneous color reader comprises a CMOS.

9. The device of claim 7, wherein the multiple-wavelength and simultaneous color reader is configured to measure absorbance quantities from the sensor.

10. The device of claim 1, further comprising a flow sensor or a volume sensor to determine flow rate of the body fluid, volume of the body fluid, and patterns of the body fluid.

11. The device of claim 1, further comprising a temperature sensor configured to measure temperature of the body fluid.

12. The device of claim 1, further comprising a barometric sensor configured to measure barometric pressure.

13. The device of claim 1, further comprising a humidity sensor configured to measure relative humidity of the body fluid.

14. The device of claim 1, further comprising a chemical sensor to sense components present in the body fluid.

15. The device of claim 14, wherein the components include oxygen and carbon dioxide.

16. The device of claim 1, further comprising a system to measure metabolic rate and respiratory quotient via oxygen consumption rate and carbon dioxide production rate.

17. The device of claim 1, wherein the device comprises a processor configured to execute a machine learning algorithm to calculate output excreted ketone concentration.

18. A method for determining ketone production rate, the method comprising:
exposing a sensor to a body fluid;
sensing, via the sensor, an excreted ketone concentration in the body fluid;
transmitting, via the sensor, a signal indicative of the excreted ketone concentration to an electronic controller;
processing, via the electronic controller, the signal;
determining, via the electronic controller, an excreted ketone production rate based on the processed signal; and
providing, via the electronic controller, an output to a user based on the excreted ketone production rate.

19. The method of claim 18, wherein sensing the excreted ketone concentration is based on pre-calibrations performed at single or several wavelengths via a multi-wavelength reader.

20. The method of claim 18, further comprising determining the excreted ketone production rate at standard conditions.

21. The method of claim 20, wherein sensing fluid ketone concentration and excreted ketone production rate at standard conditions includes using body fluid patterns, using body fluid excretion rate, or using body fluid volumes.

22. The method of claim 18, further comprising
determining, via the electronic controller, a body fluid parameter including at least one selected from the group consisting of an oxygen consumption rate, a carbon dioxide production rate, an acetone production rate, a respiratory quotient, an energy expenditure, and an acetone concentration based on the processed signal; and
providing, via the electronic controller, an output to the user based on the body fluid parameter.

23. The method of claim 18, wherein the body fluid is at least one selected from the group consisting of breath, skin, sweat, blood, urine, saliva, or excreted fluid from tissues.
